(19) United States
(12) Patent Application Publication
(10) Pub. No.: US 2020/0095532 A1
(43) Pub. Date: Mar. 26, 2020

(54) FERMENTATION MONITORING DEVICE AND METHOD FOR MONITORING A FERMENTATION PROCESS

(71) Applicant: ALCHEMA INC., Wilmington, DE (US)

(72) Inventors: Tsan-Hao YANG, Kaohsiung City (TW); Ching-Yen CHANG, New Taipei City (TW); Yu-Jui CHEN, Taipei City (TW); Yu-Wen HUANG, Tainan City (TW)

(21) Appl. No.: 16/698,822

(22) Filed: Nov. 27, 2019

Related U.S. Application Data
(63) Continuation-in-part of application No. 14/957,229, filed on Dec. 2, 2015.

(30) Foreign Application Priority Data
Feb. 4, 2015 (TW) 104103728

(51) Int. Cl. C12M 1/36; B65D 51/24; B65D 85/72; C12M 1/24; C12M 1/00; C12M 1/12

(52) U.S. Cl. CPC C12M 41/48; B65D 51/24; C12M 37/00; C12M 23/08; C12M 23/38; B65D 85/72

(57) ABSTRACT

A fermentation monitoring device is provided for monitoring a fermentation process in a bottle, and includes a detecting unit disposed at a cap body for continuously monitoring at least one environment parameter in the bottle; a weight scale disposed for detecting a weight of contents in the bottle; and a processor that, in response to receipt of an activation signal, is configured to calculate an estimated time to completion of the fermentation process, generate an alert response when a value of the environment parameter is out of a corresponding predetermined range, and determine whether the fermentation process has completed and generate a completion signal when it has.

### CROSS-REFERENCE TO RELATED APPLICATION

This application is a Continuation-in-Part Application of U.S. patent application Ser. No. 14/957,229, which was filed on Dec. 2, 2015 and which claims priority of Taiwanese Application No. 104103728, filed on Feb. 4, 2015.

### FIELD

The disclosure relates to a fermentation monitoring device and a method for monitoring a fermentation process that is to be implemented by the fermentation monitoring device.

### BACKGROUND

A fermentation process typically involves putting certain ingredients into a sealed space, in order to produce a desired product (e.g., wine, beer, yogurt, etc.). In the fermentation process, chemical reactions occur and convert specific molecules (e.g., carbohydrate) into other molecules (e.g., alcohol).

During the fermentation process, one or more environment parameters inside the sealed space may need to be closely monitored. Moreover, a time to complete the fermentation process may vary widely based on the desired product and the conditions in the sealed space.

### SUMMARY

One object of the disclosure is to provide a fermentation monitoring device that is configured to automatically monitor a fermentation process occurring in a bottle.

According to one embodiment of the disclosure, the fermentation monitoring device includes:
a cap body for sealing an opening of the bottle, the cap body and the bottle cooperating to define a fermentation space;
a detecting unit disposed at the cap body, the detecting unit including at least one sensor and being configured to continuously monitor at least one environment parameter in the fermentation space by detecting a value of the at least one environment parameter;
a communication unit connected to the processor, and configured to communicate with an electronic device;
a weight scale to be disposed at a bottom surface of the bottle and coupled to the processor for detecting a weight of contents in the bottle; and
a processor disposed at the cap body and coupled to the detecting unit.

The processor is operable, in response to receipt of an activation signal, to:
periodically and dynamically calculate an estimated time to completion of the fermentation process, based on at least the value of the at least one environment parameter as detected by the detecting unit;
determine whether the value of the at least one environment parameter is within a corresponding predetermined range;
generate an alert response when it is determined that the value of the at least one environment parameter is out of the corresponding predetermined range; and
determine whether the fermentation process has completed, and generate a completion signal when it is determined that the fermentation process has completed.

### BRIEF DESCRIPTION OF THE DRAWINGS

Other features and advantages of the disclosure will become apparent in the following detailed description of the embodiments with reference to the accompanying drawings, of which:

FIG. 1 illustrates a fermentation monitoring device according to one embodiment of the disclosure;

FIG. 2 is a bottom view of the fermentation monitoring device according to one embodiment of the disclosure;

FIG. 3 is a block diagram illustrating components of the fermentation monitoring device according to one embodiment of the disclosure;

FIG. 4 is a flow chart illustrating steps of a method for monitoring a fermentation process, according to one embodiment of the disclosure;

FIG. 5 is an exemplary chart illustrating the concentration of alcohol inside the fermentation monitoring device during different stages of a reference fermentation process;

FIGS. 6A and 6B illustrate instructions for instructing a user to put or remove specific amount of ingredients in a bottle; and

FIG. 7 illustrates an estimation of a reference fermentation time calculated using interpolation.

### DETAILED DESCRIPTION

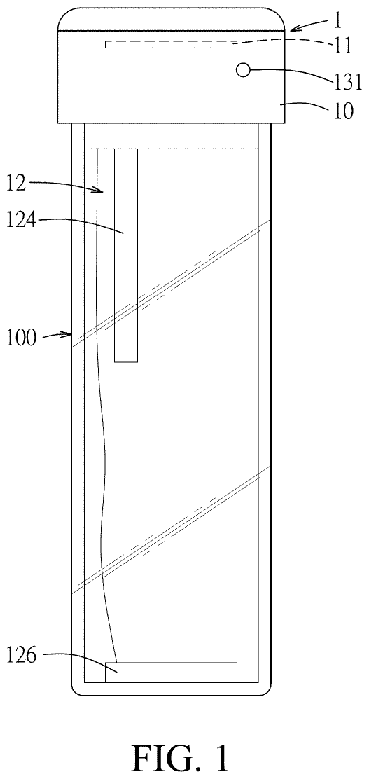

FIG. 1 illustrates a fermentation monitoring device 1 according to one embodiment of the disclosure. The fermentation monitoring device 1 may be embodied using a bottle cap that is made to seal an opening of a bottle 100. As shown in FIG. 1, when the fermentation monitoring device 1 seals the opening of the bottle 100, the fermentation monitoring device 1 and the bottle 100 cooperatively define a fermentation space.

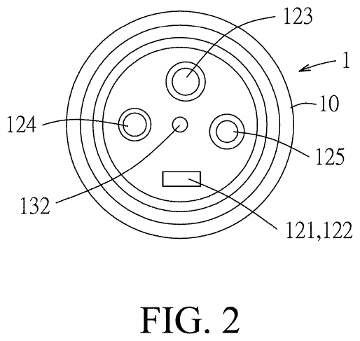

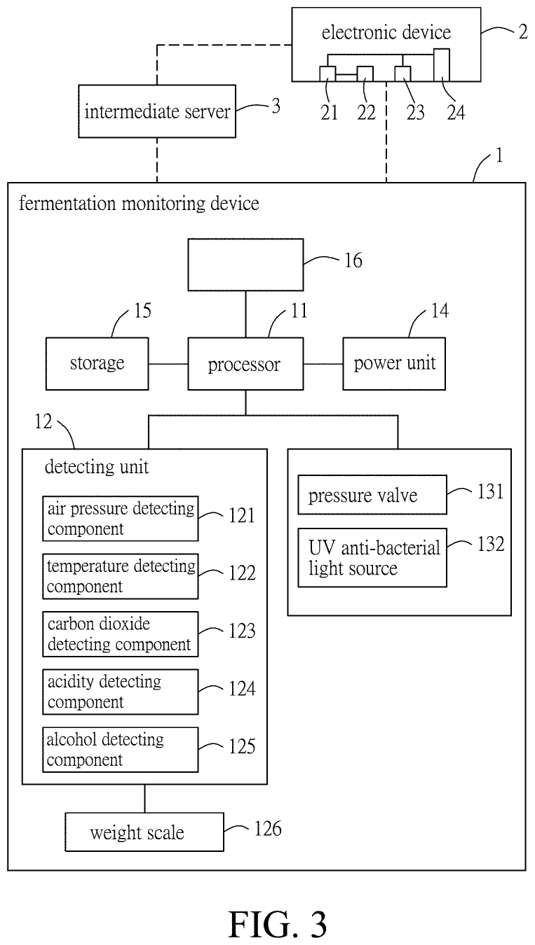

Further referring to FIGS. 2 and 3, the fermentation monitoring device 1 includes a cap body 10, a processor 11 disposed at the cap body 10, a detecting unit 12, a weight scale 126, a pressure control valve 131, an ultraviolet (UV) anti-bacterial light source 132, a power unit 14, a storage 15, and a communication unit 16.

The processor 11 may include, but not limited to, a single core processor, a multi-core processor, a dual-core mobile processor, a microprocessor, a microcontroller, a digital signal processor (DSP), a field-programmable gate array (FPGA), an application specific integrated circuit (ASIC), and/or a radio-frequency integrated circuit (RFIC), etc. Additionally, the processor 11 may be configured to include a timer function.

The detecting unit 12 is coupled to the processor 11, and includes a plurality of detecting components for respectively monitoring a plurality of environment parameters by detecting values of the environment parameters.

Referring to FIG. 3, in this embodiment, the detecting unit 12 includes an air pressure detecting component 121 for detecting a value of air pressure in the fermentation space, a temperature detecting component 122 for detecting a value of temperature in the fermentation space, a carbon dioxide detecting component 123 for detecting a value of carbon dioxide concentration in the fermentation space, an acidity detecting component 124 for detecting an acidity of a solution in the fermentation space (which may be represented by a pH value), and an alcohol detecting component 125 for detecting a value of a concentration of alcohol in the fermentation space, and the weight scale 126 is disposed at a bottom surface of the bottle 100 (see FIG. 1) and coupled to the processor 11 for detecting a weight of contents in the bottle 100. The acidity detecting component 124 extends from the cap body 10 into the fermentation space (as best depicted in FIG. 1), and the other detecting components may be disposed at the cap body 10 and be exposed, as seen in FIG. 2 (which is a bottom view of the fermentation monitoring device 1). In some embodiments, the detecting components may be embodied using respective sensor devices. For example, the air pressure detecting component 121 may be embodied using an electronic air pressure sensor. The temperature detecting component 122 may be embodied using a temperature sensor, such as an electronic temperature sensor. The carbon dioxide detecting component 123 maybe embodied using a carbon dioxide sensor, such as a non-dispersive infrared (NDIR) sensor. The acidity detecting component 124 may be embodied using an electronic pH meter for measuring hydrogen-ion activity, which may be used to indicate an acidity of a solution.

The pressure control valve 131 is disposed at the cap body 10, and may be controlled by the processor 11 to open or close in order to control the air pressure in the fermentation space.

The UV anti-bacterial light source 132 is disposed at the cap body 10, and may be controlled to activate in order to irradiate the fermentation space with ultraviolet light.

The power unit 14 provides the power required for operation of the other components of the fermentation monitoring device 1. In this embodiment, the power unit 14 is, for example, a rechargeable battery set and may be recharged wirelessly via an external power source.

The storage 15 is coupled to the processor 11, and stores information regarding the environment parameters therein. Specifically, the storage 15 may store a database that contains, for each of the environment parameters, predetermined range associated with the environment parameter.

The processor 11 may be capable of communicating with an electronic device 2, and controlled by the electronic device 2 perform various operations. The communication between the processor 11 and the electronic device 2 may be done using a local area network (LAN), a BLUETOOTH® communication, or via an intermediate server 3. The electronic device 2 may be embodied using a smartphone, a laptop computer, or a tablet computer, etc., and includes a processor 21, a display screen 22, a communication component 23, and a storage component 24.

The processor 11 is operable to implement a method for monitoring a fermentation process that is to be carried out in the bottle 100 (i.e., the fermentation space). That is to say, when a user intends to produce a specific product by conducting a fermentation process in the bottle 100, the user may place the relevant ingredients inside the bottle 100, seal the opening of the bottle 100 using the fermentation monitoring device 1, and activate the processor 11 of the fermentation monitoring device 1 to start monitoring the fermentation process. For example, the user may operate the electronic device 2 to send an activation signal to the fermentation monitoring device 1, or push a start button (not shown) on the fermentation monitoring device 1 in order to activate the processor 11.

It is noted that, prior to implementation of the method, the database may be constructed to include a plurality of template models. Each of the template models is associated with a specific product that can be made via a fermentation process, includes a recipe of the product, and may include fermentation data associated with different stages of the fermentation process of the product. The recipe may include one or more ingredients, a weight of each of the one or more ingredients, and, in the case that a plurality of ingredients are involved, a specific order in which the ingredients are to be put into the bottle 100. The database may then be stored in the storage 15.

For each of the template models, the fermentation data included in the template model may be generated by performing a reference fermentation process in the fermentation space for the specific product (e.g., a specific kind of wine) associated with the template model. During the reference fermentation process, the detecting unit 12 is activated to detect the environment parameters within the fermentation space.

In one example, a specific environment parameter (e.g., a concentration of alcohol) inside the bottle 100 may be recorded during the reference fermentation process for later references. The concentration of alcohol maybe periodically (e.g., once every 10 minutes) calculated by obtaining a detected weight of the contents in the bottle 100, and calculating the concentration of alcohol using an equation and the obtained weight of the contents.

Specifically, it is noted that the exemplary fermentation reaction involves sugar (with the molecular formula C₆H₁₂O₆) being converted into alcohol and carbon dioxide (with presence of zymase), as represented by the following chemical equation:

C₆h₁₂O₆→2C₂H₅OH+2CO₂

where the sugar may be in a solid phase, the alcohol may be in a liquid phase, and the carbon dioxide may be in a gas phase.

As such, as more alcohol is being generated, an amount of sugar is reduced, and more carbon dioxide is also generated, slightly reducing the overall weight detected by the weight scale 126. Afterward, a concentration of alcohol may be calculated based on a reduction in the overall weight as detected by the weight scale 126. Specifically, according to the above equation, when a specific amount of carbon dioxide is generated (in the unit of mole (mol)), a same amount of alcohol is generated. That is to say, the reduction in the overall weight may be used to determine the amount of carbon dioxide generated (e.g., a reduction of 44 grams in the overall weight is equivalent to one mole of carbon dioxide being generated). Therefore, the amount of alcohol, and in turn, the weight of generated alcohol and the concentration of alcohol may be deduced based on the reduction in the overall weight. It is noted that a sweetness of the product in the bottle 100 may also be reduced during the reference fermentation process, and may be deduced using the above manner.

Additionally, the temperature detecting component 122 is also configured to obtain the value of temperature in the fermentation space periodically.

That is to say, in this example, a set of parameters in the form of (Mod_Alc[k], Mod_Temp[k], T[k]) may be obtained. Specifically, Mod_Alc[k] represents a calculated concentration of the alcohol at a k^{th} time instance, Mod_Temp[k] represents an obtained value of temperature in the fermentation space at the k^{th} time instance, and T[k] represents a elapsed time length since the start of the fermentation process at the k^{th} time instance.

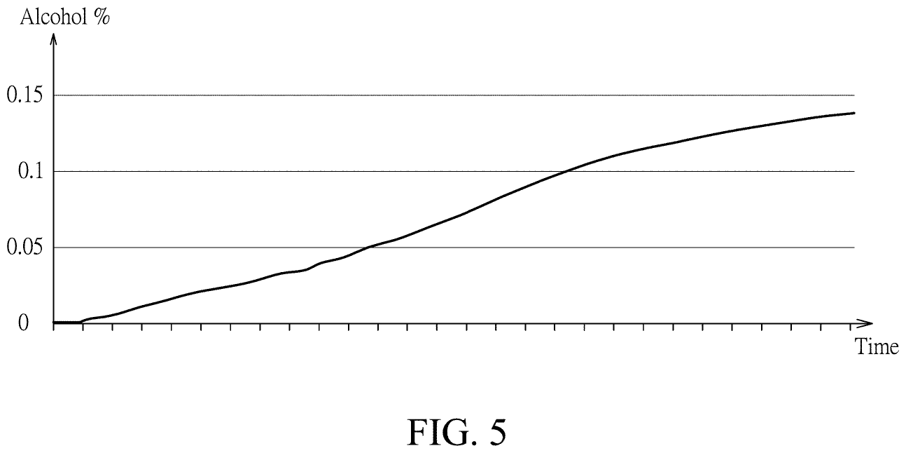

After the reference fermentation process is completed, the concentration of alcohol inside the bottle 100 at different stages of the reference fermentation process may be calculated by the processor 11. FIG. 5 is an exemplary graph illustrating the concentration of alcohol inside the bottle 100 at different stages of the reference fermentation process of sake, a Japanese rice wine. The content included in FIG. 5 may be stored in one of the template models that is associated with sake as the fermentation data.

Furthermore, in some examples, the processor 11 is configured to calculate an average value of temperature Temp[avg] obtained in the fermentation space throughout the reference fermentation process. The average value of temperature Temp[avg] may also be stored in the one of the template models that is associated with sake.

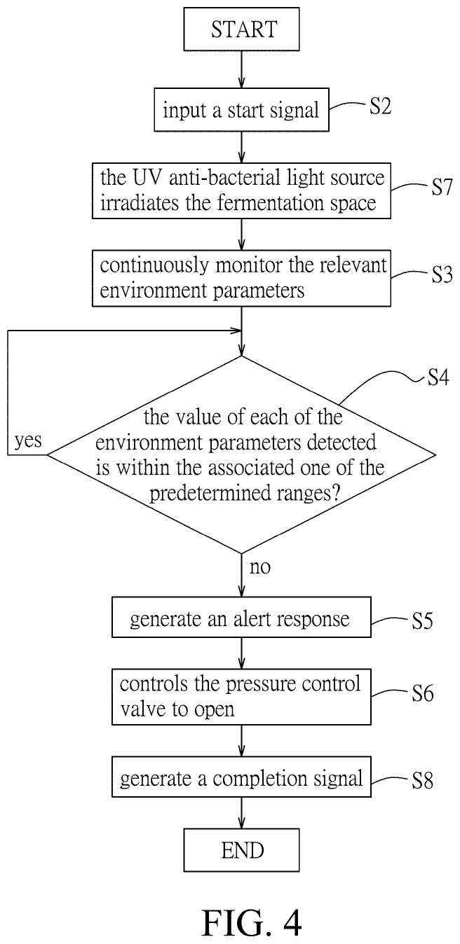

FIG. 4 is a flow chart illustrating steps of a method for monitoring the fermentation process, according to one embodiment of the disclosure.

Before the fermentation process begins, the user may operate the electronic device 2 to transmit a signal to the processor 11. Specifically, the user may operate the electronic device 2 to execute a software application stored in the storage component 24. In one example where the electronic device 2 is embodied using a mobile phone, the application may be executed by the user clicking on an icon displayed on the display screen 22.

When executed by the processor 21 of the electronic device 2, the software application causes the processor 21 to control the communication component 23 to establish communication with the fermentation monitoring device 1. Afterward, the environment parameters detected by the fermentation monitoring device 1 may be transmitted to the electronic device 2 for calculation and then display for view by the user.

In another embodiment, after bringing the electronic device 2 to a place within a certain distance from the fermentation monitoring device 1, the user may operate the fermentation monitoring device 1 (e.g., by pressing a button on the fermentation monitoring device 1), so the communication unit 16 of the fermentation monitoring device 1 establishes a connection with the electronic device 2.

Then, the processor 21 of the electronic device 2 controls the display screen 22 of the electronic device 2 to display a menu having a plurality of products for allowing the user to select one of the products that is intended to be produced via the fermentation process. In some embodiments, the user may also operate the electronic device 2 to input one or more target characteristics for the selected product (e.g., a preferred concentration of alcohol, sweetness, etc.).

After the user selects one of the products, the processor 21 of the electronic device 2 accesses the database to obtain information contained in one of the template models that is associated with the selected one of the products.

Then, the processor 21 may further control the display screen 22 to display the recipe for the selected one of the products. It is noted that the content of the recipe may be adjusted based on the target characteristics inputted by the user.

It is noted that there may be cases in which some of the products do not have corresponding fermentation data (that is, no corresponding reference fermentation has been performed with respect to some of the products). In such cases, the processor 21 may select one of the template models with similar ingredients or a default template model and use the fermentation data thereof for the upcoming fermentation process.

After the ingredient(s) is(are) put in the bottle 100 by the user, the fermentation monitoring device 1 is used to seal the bottle 100 thereby defining the fermentation space.

It is noted that the processor 21 may employ the weight detected by the weight scale 126 to verify whether the ingredient(s) is(are) put in the bottle 100 (i.e., whether the weight detected by the weight scale 126 equals or substantially equals a net weight of ingredient(s) listed in the recipe).

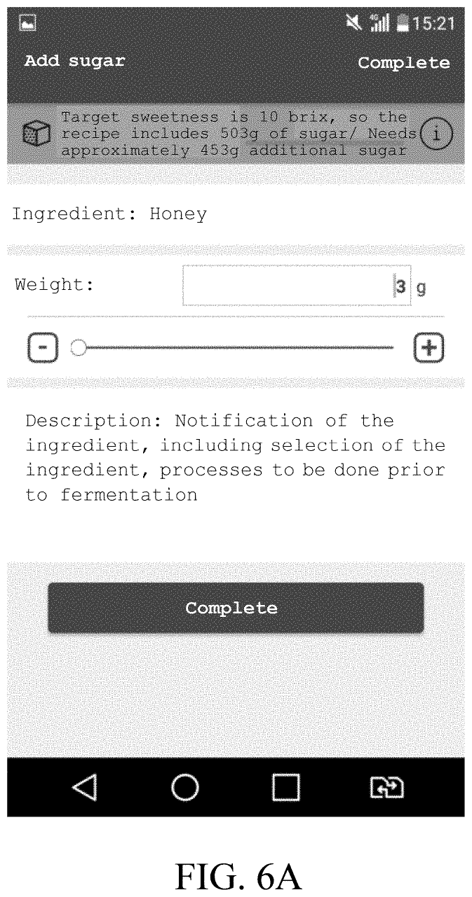

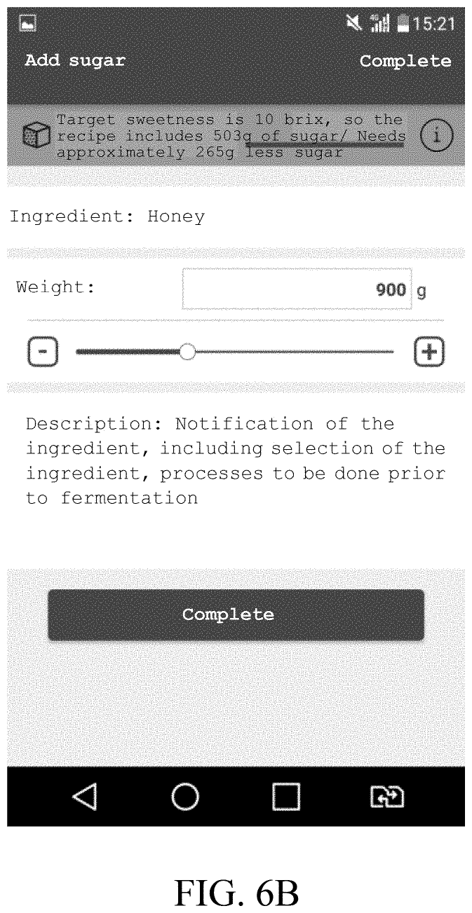

In one embodiment, producing sake requires multiple ingredients, and the processor 21 may control the display screen 22 to display a series of instructions for instructing the user to put the ingredients sequentially into the bottle 100. For example, FIG. 6A illustrates one instruction instructing the user to put a designated amount of sugar (e.g., 503 grams). Afterward, a difference between the weight detected by the weight scale 126 at a reference time instance the instruction is displayed and the weight currently detected by the weight scale 126 at a current time instance after the reference time instance may be used to serve as an amount of sugar that has been put in the bottle 100. When the difference is lower than the designated amount, the processor 21 may adjust the instruction for instructing the user to put an amount needed to reach the designated amount (FIG. 6A). On the other hand, when the difference is higher than the designated amount, the processor 21 may adjust the instruction for instructing the user to remove an excess amount of sugar needed to match the designated amount (FIG. 6B).

After the ingredients are put in the bottle 100, to initiate the method for monitoring the fermentation process, in step S2, the user operates the electronic device 2 to input a start signal (by, for example, pressing a button on the electronic device 2). In response, the electronic device 2 communicates with the fermentation monitoring device 1 and transmits the start signal to the fermentation monitoring device 1, indicating that monitoring of the fermentation process is to be started and indicating the selected one of the products. Based on the selected product, the processor 11 of the fermentation monitoring device 1 may determine which ones of the environment parameters are to be monitored, and adjust the predetermined ranges associated with to-be-monitored ones of the environment parameters based on the selected product so that the predetermined ranges are appropriate for the selected product.

In an example where the selected one of product is sake, the to-be-monitored environment parameters may include concentration of alcohol and temperature of the fermentation space.

A predetermined range for the value of each of the concentration of alcohol and the temperature may be determined based on information included in the fermentation data of the template model corresponding to sake, and an elapsed time of the fermentation process. For example, the predetermined range for the value of the concentration of alcohol may be defined by a highest point and a lowest point of the concentration of alcohol recorded in the reference fermentation process for producing sake. The predetermined range of the value of the temperature may be defined by a highest point and a lowest point of temperature recorded in the reference fermentation process for producing sake.

In step S3, the detecting unit 12 is activated to continuously monitor the relevant environment parameters. It is noted that the calculation of the value of the concentration of alcohol may be implemented in a manner similar to that as described in the reference fermentation process.

In step S3, the processor 11 may further be configured to calculate an estimated time to completion indicating when the fermentation process is estimated to complete, based on the values of the environment parameters that are most recently detected. In this embodiment, the processor 11 calculates the estimated time to completion, and transmits a notification of the estimated time to completion to the electronic device 2 so as to enable the electronic device 2 to display the estimated time to completion on the display screen 22, and to count down the time to completion.

Specifically, in the example where the selected product is sake, the processor 11 may first determine a reference fermentation time Mod_Y required to produce sake by the fermentation process from the template model, based on the inputted target characteristics of sake (e.g., a preferred value of the concentration of alcohol, or a default number if no target characteristic is inputted). In this example, the concentration of alcohol reaches the preferred concentration in the reference fermentation process when the reference fermentation time has just passed.

It is noted that since in the reference fermentation process, values of the environment parameters were obtained periodically (e.g., once every 10 minutes), the preferred concentration may not be identical to any value obtained in the reference fermentation process. In such a case, an estimation of the reference fermentation time may be calculated using interpolation.

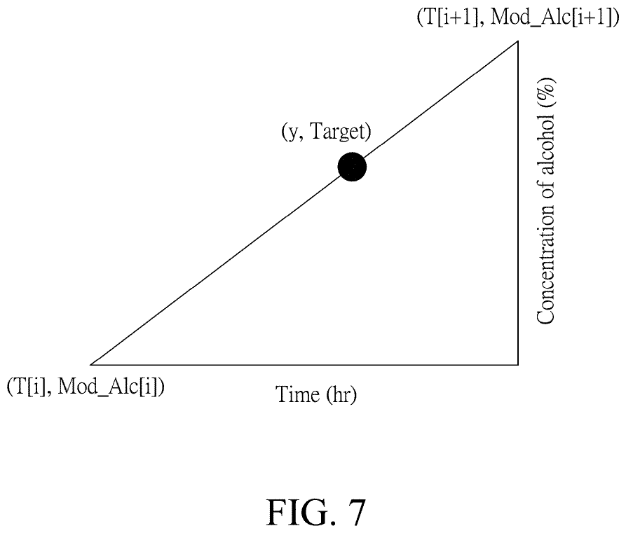

That is to say, the processor 11 may first find, in the template model, two values of the concentration of alcohol that are obtained in succession (i.e., Mod_Alc[i] and Mod_Alc [i+1]) that satisfy a condition that a value of the preferred concentration is between the two concentrations (i.e., Mod_Alc[i]<target %<Mod_Alc[i+1], target %. A representing the preferred concentration). Then, the reference fermentation time should be between two elapsed times associated with the two values of the concentration of alcohol (i.e. , T[i]<Mod_Y<T[i+1]) as exemplified in FIG. 7.

Afterward, the processor 11 obtains the to-be-monitored environment parameters (i.e., concentration of the alcohol Alc[n], current temperature Temp[n] and a current elapsed time T[n]).

Using the to-be-monitored environment parameters thus obtained, the processor 11 is configured to determine a progress of the fermentation process by calculating an expected elapsed time (Y) based on the current value of the concentration of alcohol. Specifically, the processor 11 may find, in the template model, the time elapsed in the reference fermentation process for the concentration of alcohol to reach the preferred concentration, and this time thus determined is then used to serve as the expected elapsed time (Y) Interpolation may also be employed in a manner similar to that used for determining the reference fermentation time.

Then, the processor 11 determines a time ratio (Rₜᵢₘₑ) of the current elapsed time T[n] to the expected elapsed time (Y), and calculates an expected fermentation time (MOD_Y*Rₜᵢₘₑ) for the fermentation process. The estimated time to completion may be calculated by subtracting the current elapsed time T[n] from the expected fermentation time (MOD_Y*Rₜᵢₘₑ).

In some embodiments, the processor 11 further employs the temperature for adjusting the estimated time to completion. Specifically, the processor 11 may determine a temperature ratio (Rₜₑₘₚ) of the current temperature Temp[n] to the average value of temperature Temp[avg] corresponding to the reference fermentation process. Then, an adjusted estimated time to completion may be calculated by multiplying the estimated time to completion by the temperature ratio (Rₜₑₘₚ).

It is worth noting that in one embodiment, the processor 11 periodically estimates a time to completion. This is done because the values of the environment parameters in the fermentation space may change rapidly during the fermentation process, and by dynamically estimating and updating the time to completion, a more accurate estimation may yield. Specifically, the processor 11 may perform the above-described operations to calculate the estimated time to completion.

In some cases, when the value(s) of one or more particular environment parameters reaches a specific level (e.g., concentration of alcohol reaches the preferred concentration of alcohol inputted by the user), the processor 11 may determine that the fermentation process is complete.

In step S4, during the fermentation process, the processor 11 determines whether the value of each of the environment parameters detected in step S3 is within the associated one of the predetermined ranges.

When it is determined that the value of one of the monitored environment parameters is out of the associated one of the predetermined ranges, the processor 11 generates an alert response in step S5.

For example, a pH value may be detected by the acidity detecting component 124 periodically. When the pH value is deemed to be too high (e.g., 8) or too low (e.g., 3) in step S4, the flow proceeds to step 35 to generate the alert response.

The alert response may be transmitted to the electronic device 2 so as to notify the user, such that the user may perform various procedures to rectify the value(s) of the environment parameter(s).

The processor 11 may automatically address some of the issues associated with the environment parameter(s) and determined in step S4. For example, in step S6, when it is determined that the air pressure in the fermentation space is larger than an upper limit of the associated predetermined range, the processor 11 controls the pressure control valve 131 to open so as to decrease the air pressure in the fermentation space.

It is noted that, at any stage of the fermentation process, the UV anti-bacterial light source 132 may be activated by the processor 11 in order to irradiate the fermentation space (step S7). In this embodiment, the UV anti-bacterial light source 132 maybe activated right after the monitoring of the fermentation process has started.

In step S8, when the time to completion as estimated by the processor 11 becomes zero and/or the processor 11 determines that the fermentation process is complete (e.g., when the concentration of alcohol reaches the preferred concentration), the processor 11 may generate a completion signal and transmit the completion signal to the electronic device 2. Alternatively, when the countdown of the time to completion conducted by the electronic device 2 reaches zero, the electronic device 2 generates an alert to notify the user that the fermentation process has completed. As such, the user may be notified to remove the product from the bottle 100 at a suitable time.

To sum up, the fermentation monitoring device 1 and the method for monitoring the fermentation process as described in the disclosure are configured to monitor the environment parameters in the fermentation space, and to provide an alert response when the values of the environment parameters are out of the respective predetermined ranges. Moreover, the fermentation monitoring device 1 is configured to estimate the time to completion and to address some of the issues regarding the environment parameters automatically, and is therefore able to provide the user with more user-friendly environment for executing the fermentation process.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A fermentation monitoring device for monitoring a fermentation process occurring in a bottle, said fermentation monitoring device comprising:
a cap body for sealing an opening of the bottle, said cap body and the bottle cooperating to define a fermentation space;
a detecting unit disposed at said cap body, said detecting unit including at least one sensor and being configured to continuously monitor at least one environment parameter in the fermentation space by detecting a value of the at least one environment parameter;
a communication unit connected to said processor, and configured to communicate with an electronic device;
a weight scale to be disposed at a bottom surface of the bottle and coupled to said processor for detecting a weight of contents in the bottle; and
a processor disposed at said cap body and coupled to said detecting unit, said processor being operable, in response to receipt of an activation signal, to
periodically and dynamically calculate an estimated time to completion of the fermentation process, based on at least the value of the at least one environment parameter as detected by said detecting unit,
determine whether the value of the at least one environment parameter is within a corresponding predetermined range,
generate an alert response when it is determined that the value of the at least one environment parameter is out of the corresponding predetermined range, and
determine whether the fermentation process has completed, and generate a completion signal when it is determined that the fermentation process has completed.

2. The fermentation monitoring device of claim 1, wherein the at least one environment parameter that is monitored by said at least one sensor includes one of an air pressure in the fermentation space, a temperature in the fermentation space, a carbon dioxide concentration in the fermentation space, an alcohol concentration in the fermentation space, and combinations thereof.

3. The fermentation monitoring device of claim 1, wherein said processor is operable, in response to receipt of a user input of a product that is intended to be produced by the fermentation process, to adjust the predetermined range based on the user input of the product.

4. The fermentation monitoring device of claim 1, further comprising a pressure control valve that is coupled to and controlled by said processor, wherein:
the at least one environment parameter monitored by said at least one sensor includes an air pressure in the fermentation space; and
when it is determined that the value of the air pressure in the fermentation space is larger than an upper limit of the corresponding predetermined range, said processor is configured to control said pressure control valve to open so as to decrease the value of the air pressure in the fermentation space.

5. The fermentation monitoring device of claim 1, wherein said processor is capable of communicating with an electronic device, and is programmed to:
periodically estimate a time to completion based on the value of the at least one environment parameter that is most recently detected, the time to completion indicating when the fermentation process is estimated to complete; and
transmit a notification of the time to completion to the electronic device so as to enable the electronic device to count down the time to completion.

6. The fermentation monitoring device of claim 5, wherein said processor is further programmed to:
estimate the time to completion further based on the weight of contents in the bottle as detected by said weight scale.

7. The fermentation monitoring device of claim 6, wherein, prior to a start of the fermentation process, said processor is programmed to obtain a template model that is associated with a product and that includes fermentation data associated with different stages of a of the product, and is programmed to estimate the time to completion by:
during the fermentation process, determining a reference fermentation time and a current elapsed time of the fermentation process;
calculate a concentration of the product based on the weight of content in the bottle detected by said weight scale;
calculating a ratio of the current elapsed time an expected elapsed time in the reference fermentation process;
calculating an expected fermentation time for the fermentation process based on the reference fermentation time and the ratio; and
estimating the time to completion based on the expected fermentation time and the current elapsed time.

8. The fermentation monitoring device of claim 7, wherein said processor is programmed to estimate the time to completion further by:
determining an average value of temperature in the reference fermentation process;
determining a temperature ratio of the current temperature to the average value of temperatures; and
calculating an adjusted estimated time to completion by multiplying the estimated time to completion by the temperature ratio.
